(19) United States
(12) Patent Application Publication
(10) Pub. No.: US 2022/0309397 A1
(43) Pub. Date: Sep. 29, 2022

(54) PREDICTION MODEL RE-LEARNING DEVICE, PREDICTION MODEL RE-LEARNING METHOD, AND PROGRAM RECORDING MEDIUM

(71) Applicant: NEC Corporation, Minato-ku, Tokyo (JP)

(72) Inventors: So YAMADA, Tokyo (JP); Riki ETO, Tokyo (JP); Junko WATANABE, Tokyo (JP); Hiromi SHIMIZU, Tokyo (JP); Hidetaka HANE, Tokyo (JP); Shigeo KIMURA, Tokyo (JP); Wataru FUJII, Tokyo (JP); Tomoyuki KAWABE, Tokyo (JP)

(73) Assignee: NEC Corporation, Minato-ku, Tokyo (JP)

(21) Appl. No.: 17/618,045

(22) PCT Filed: Jun. 19, 2019
(86) PCT No.: PCT/JP2019/024338

§ 371 Date: Dec. 10, 2021

(51) Int. Cl. G06N 20/00; G01N 33/02

(52) U.S. Cl. CPC G06N 20/00; G01N 33/025

(57) ABSTRACT

To mitigate degradation in the accuracy of a prediction model by re-learning the prediction model with consideration given to the characteristics of a detection value of a sensor.

This prediction model re-learning device comprises: a calculation unit that, on the basis of data related to smell detection by a sensor, calculates an index for determining whether or not to re-learn a prediction model for smell; and a re-learning unit that re-learns the prediction model in cases where the calculated index satisfies a predetermined condition.

### TECHNICAL FIELD

The present invention relates to a prediction model re-learning device that re-learns a prediction model, a prediction model re-learning method, and a program recording medium.

### BACKGROUND ART

It is known that prediction precision of a prediction model deteriorates over time due to a change in environment or the like.

Therefore, PTL 1 discloses a technique of re-learning a prediction model based on an evaluation index for evaluating the precision of the prediction model.

PTL 2 discloses a technique of re-learning a prediction model for identifying a smell every time measurement of five samples is completed.

### CITATION LIST

### Patent Literature

[PTL 1] JP WO 2016/151618 A

[PTL 2] JP 1992-186139 A

### SUMMARY OF INVENTION

### Technical Problem

By the way, a sensor that detects a smell has a characteristic that a behavior of a detection value of the sensor changes when a measurement environment such as temperature or humidity changes.

However, the evaluation index described in PTL 1 does not consider the above characteristic. Therefore, in the technique described in PTL 1, there is a case where the precision deterioration of the prediction model using a detection value of a sensor is not mitigated.

The technique described in PTL 2 does not consider the above characteristic because re-learning is performed every time the measurement of five samples is completed. Therefore, the technique described in PTL 2 may not mitigate precision deterioration of the prediction model.

Therefore, an object of the present invention is to mitigate precision deterioration of a prediction model.

### Solution to Problem

A prediction model re-learning device according to the present invention includes calculation means for calculating an index for determining whether to re-learn a prediction model for a smell based on data related to smell detection by a sensor; and re-learning means for re-learning the prediction model in a case where the calculated index satisfies a predetermined condition.

A prediction model re-learning method according to the present invention calculates an index for determining whether to re-learn a prediction model for a smell based on data related to smell detection by a sensor; and re-learns the prediction model in a case where the calculated index satisfies a predetermined condition.

A program recording medium according to the present invention causes a computer to execute: processing of calculating an index for determining whether to re-learn a prediction model for a smell based on data related to smell detection by a sensor; and processing of re-learning the prediction model in a case where the calculated index satisfies a predetermined condition.

### Advantageous Effects of Invention

The present invention has an effect of mitigating precision deterioration of a prediction model.

### BRIEF DESCRIPTION OF DRAWINGS

FIG. 1 is a diagram illustrating a sensor 10 for obtaining data acquired by a prediction model re-learning device 2000.

FIG. 2 is conceptual diagrams of a prediction model.

FIG. 3 is a diagram illustrating a functional configuration of the prediction model re-learning device 2000 of a first example embodiment.

FIG. 4 is a diagram illustrating a computer for implementing the prediction model re-learning device.

FIG. 5 is a diagram illustrating a flow of processing executed by the prediction model re-learning device 2000 of the first example embodiment.

FIG. 6 is a table illustrating smell data stored in a storage unit 2010.

FIG. 7 is a table illustrating a correspondence relationship between a prediction model and training data stored in the storage unit 2010.

FIG. 8 is a table illustrating a condition for re-learning stored in the storage unit 2010.

FIG. 9 is a diagram illustrating a flow of processing of a calculation unit 2020.

FIG. 10 is a diagram illustrating a flow of processing of a re-learning unit 2030.

FIG. 11 is a diagram illustrating a functional configuration of a prediction model re-learning device 2000 of a second example embodiment.

FIG. 12 is a diagram illustrating a flow of processing executed by the prediction model re-learning device 2000 of the second example embodiment.

FIG. 13 is a table illustrating smell data stored in a storage unit 2010 in the second example embodiment.

FIG. 14 is a table illustrating a condition stored in the storage unit 2010 and used for determining whether a re-learning unit 2030 performs re-learning.

FIG. 15 is a diagram illustrating a contribution value for each feature constant to time-series data.

FIG. 16 is a diagram illustrating a flow of processing of a calculation unit 2050.

FIG. 17 is a diagram illustrating a functional configuration in a modification of the second example embodiment.

FIG. 18 is a diagram illustrating a functional configuration of a prediction model re-learning device 2000 of a third example embodiment.

FIG. 19 is a diagram illustrating a flow of processing executed by the prediction model re-learning device 2000 of the third example embodiment.

FIG. 20 is a diagram illustrating a flow of processing of an update determination unit 2070.

### EXAMPLE EMBODIMENT

### First Example Embodiment

Hereinafter, a first example embodiment according to the present invention will be described.

<Sensor>

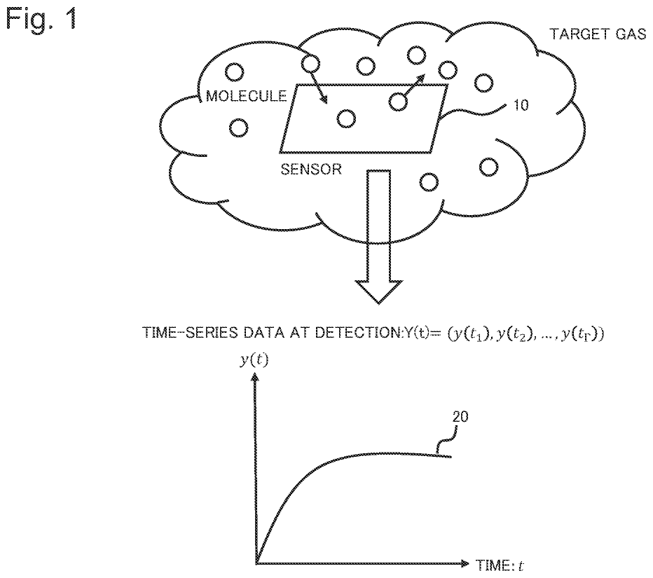

A sensor used in the present example embodiment will be described. FIG. 1 is a diagram illustrating a sensor 10 that detects a smell and time-series data obtained by the sensor 10 detecting the smell.

The sensor 10 is a sensor having a receptor to which a molecule is attached, and a detection value changes according to attachment or detachment of the molecule at the receptor. Note that a gas sensed by the sensor 10 is referred to as a target gas. Furthermore, the time-series data of the detection value output from the sensor 10 is referred to as time-series data 20. Here, as necessary, the time-series data 20 is also referred to as Y, and the detection value at time t is also referred to as y(t). Y is a vector in which y (t) is listed.

For example, the sensor 10 may be a membrane-type surface stress sensor (MSS). The MSS has a sensory membrane to which the molecule is attached as the receptor, and stress generated in a support member of the sensory membrane changes according to the attachment or detachment of the molecule to or from the sensory membrane. The MSS outputs the detection value based on this change in stress. Note that the sensor 10 is not limited to the MSS, and may be any sensor as long as the sensor outputs the detection value based on a change in a physical quantity related to viscoelasticity or a dynamic characteristic (mass, inertia moment, or the like) of a member of the sensor 10, which occurs according to the attachment or detachment of the molecule to or from the receptor. Various types of sensors such as cantilever-type, membrane-type, optical-type, piezo-, and vibration-response sensors can be adopted.

### Prediction Model

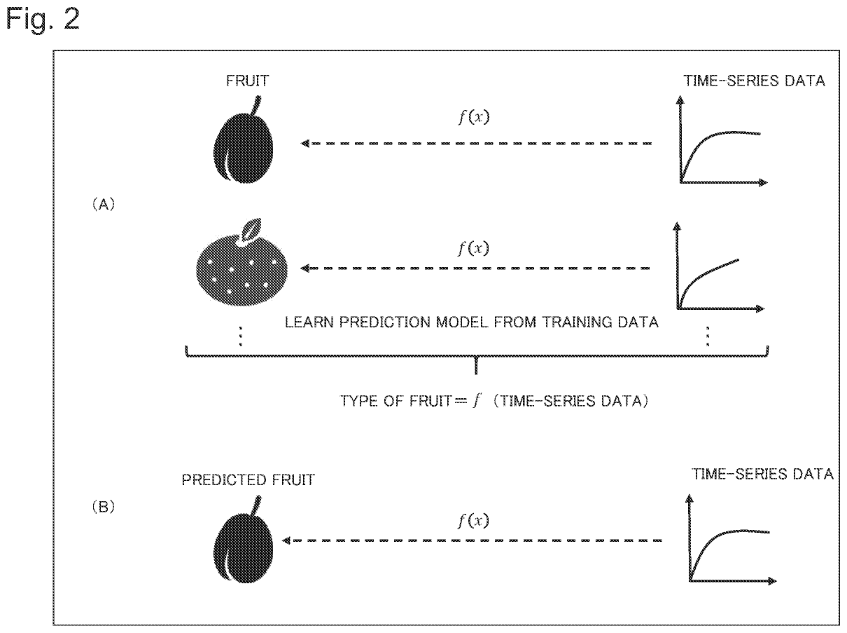

The prediction model used in the present example embodiment will be described. FIG. 2 is conceptual diagrams of a prediction model. Here, a prediction model for predicting a type of fruit from the time-series data of the detection value output from the sensor 10 will be described as an example. FIG. 2(A) illustrates a phase of learning the prediction model. In FIG. 2(A), the prediction model is learned using a combination of the type of a certain fruit (for example, apple) and the time-series data 20 of the detection value output from the sensor 10 as training data. FIG. 2(B) illustrates a phase of using the prediction model. In FIG. 2(B), the prediction model receives, as an input, time-series data acquired from a fruit of an unknown type, and outputs the type of the fruit as a prediction result.

Note that, in the example embodiment to be described below, the prediction model is not limited to the model that predicts the type of fruit. The prediction model is only required to output the prediction result based on the time-series data of the detection value output from the sensor 10. For example, the prediction model may predict the presence or absence of a specific disease from exhalation of a person, may predict the presence or absence of a harmful substance from a smell in a house, or may predict abnormality of factory equipment from a smell in the factory.

### Example of Functional Configuration of Prediction Model Re-Learning Device 2000

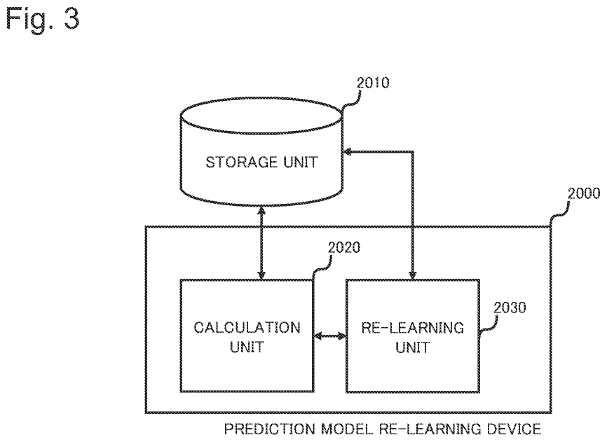

FIG. 3 is a diagram illustrating a functional configuration of a prediction model re-learning device 2000 of the first example embodiment. The prediction model re-learning device 2000 includes a calculation unit 2020 and a re-learning unit 2030. The calculation unit 2020 acquires data (hereinafter smell data) related to smell detection by the sensor from a storage unit 2010, and calculates an index for determining whether to re-learn the prediction model. The re-learning unit 2030 determines whether to re-learn the prediction model based on the index calculated by the calculation unit 2020. In the case of determining to re-learn the prediction model, the re-learning unit 2030 re-learns the prediction model.

### Hardware Configuration of Prediction Model Re-Learning Device 2000

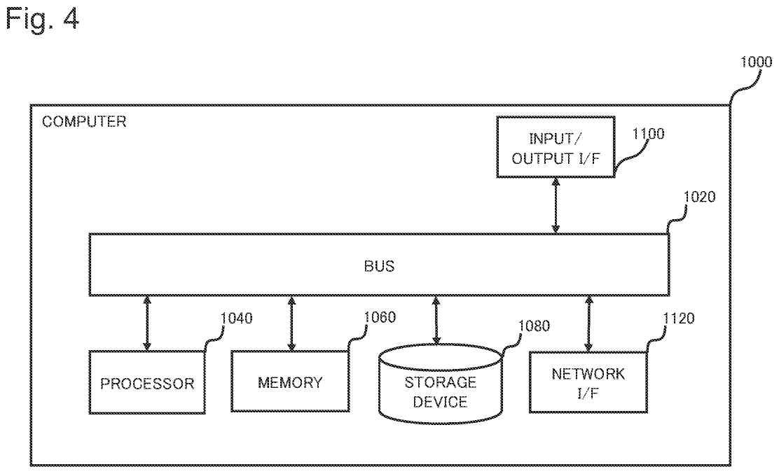

FIG. 4 is a diagram illustrating a computer for implementing the prediction model re-learning device 2000 illustrated in FIG. 3. A computer 1000 is any computer. For example, the computer 1000 is a stationary computer such as a personal computer (PC) or a server machine. Alternatively, for example, the computer 1000 is a portable computer such as a smartphone or a tablet terminal. The computer 1000 may be a dedicated computer designed for implementing the prediction model re-learning device 2000 or may be a general-purpose computer.

The computer 1000 includes a bus 1020, a processor 1040, a memory 1060, a storage device 1080, an input/output interface 1100, and a network interface 1120. The bus 1020 is a data transmission path for the processor 1040, the memory 1060, the storage device 1080, the input/output interface 1100, and the network interface 1120 to transmit and receive data to and from one another. Note that the method of connecting the processor 1040 and the like to one another is not limited to the bus connection.

The processor 1040 is various processors such as a central processing unit (CPU), a graphics processing unit (GPU), and a field-programmable gate array (FPGA). The memory 1060 is a main storage device implemented by using a random access memory (RAM) or the like. The storage device 1080 is an auxiliary storage device implemented by using a hard disk, a solid-state drive (SSD), a memory card, a read only memory (ROM), or the like.

The input/output interface 1100 is an interface for connecting the computer 1000 and an input/output device. For example, an input device such as a keyboard and an output device such as a display device are connected to the input/output interface 1100. In addition, for example, the sensor 10 is connected to the input/output interface 1100. Note that the sensor 10 is not necessarily directly connected to the computer 1000. For example, the sensor 10 may store the acquired data in a storage device shared with the computer 1000.

The network interface 1120 is an interface for connecting the computer 1000 to a communication network. The communication network is, for example, a local area network (LAN) or a wide area network (WAN). The method of connecting the network interface 1120 to the communication network may be wireless connection or wired connection.

The storage device 1080 stores program modules that implement the functional configuration units of the prediction model re-learning device 2000. The processor 1040 reads the program modules to the memory 1060 and executes the program modules, thereby implementing the functions related to the program modules.

### Flow of Processing

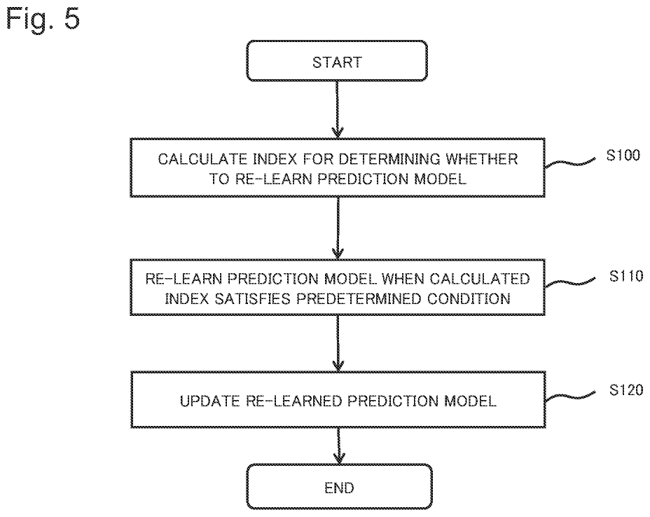

FIG. 5 is a diagram illustrating a flow of processing executed by the prediction model re-learning device 2000 of the first example embodiment. The calculation unit 2020 calculates the index of whether to re-learn the prediction model from the smell data (S100). The re-learning unit 2030 re-learns the prediction model based on the calculated index (S110). The re-learning unit 2030 stores the re-learned prediction model in the storage unit 2010 to update the prediction model (S120).

### Information Stored in Storage Unit 2010

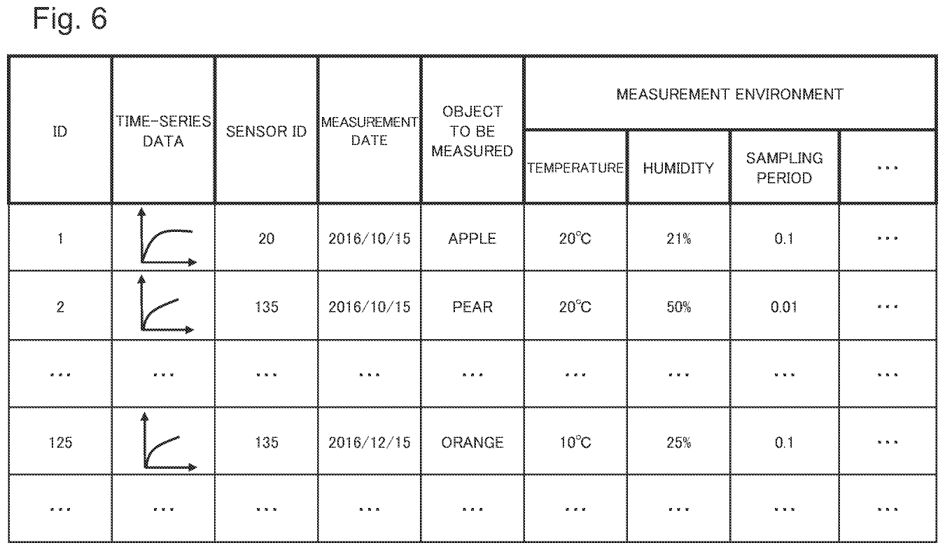

Information stored in the storage unit 2010 will be described. FIG. 6 is a table illustrating smell data stored in the storage unit 2010.

Each record in FIG. 6 is related to the smell data. Each smell data includes, for example, an ID for identifying the smell data, time-series data obtained by the sensor 10 detecting the smell a sensor ID for identifying the sensor 10 that has detected the smell, a measurement date, an object to be measured, and a measurement environment.

The measurement date may be, for example, a date on which the target gas has been injected into the sensor 10 or a date on which the acquired smell data has been stored in the storage unit 2010. Note that the measurement date may be measurement date and time including a measurement time.

The measurement environment is information related to the environment when the smell is measured. As illustrated in FIG. 6, the measurement environment includes, for example, temperature, humidity, and a sampling period of the environment in which the sensor 10 is installed.

The sampling period indicates a reciprocal for measuring the smell and is expressed as Δt [s] or a sampling frequency [Hz] using an inverse number of Δt [s]. For example, the sampling period is 0.1 [s], 0.01 [s], or the like.

When the smell is measured by alternately injecting a sample gas and a purge gas to the sensor 10, an injection time of the sample gas and the purge gas may be set as the sampling period. Here, the sample gas is a target gas in FIG. 1. The purge gas is a gas (for example, nitrogen) for removing the target gas attached to the sensor 10. For example, the sensor 10 can measure data when the sample gas is injected for five seconds and the purge gas is injected for five seconds.

The measurement environment such as the above-described temperature, humidity, and sampling period may be acquired by, for example, a meter provided inside or outside the sensor 10, or may be input from a user.

In the present example embodiment, the temperature, humidity, and sampling period have been described as examples of the measurement environment, but other examples of the measurement environment include information of a distance between an object to be measured and the sensor 10, the type of the purge gas, a carrier gas, the type of the sensor (for example, the sensor ID), a season at the time of measurement, an atmospheric pressure at the time of measurement, an atmosphere (for example, CO₂ concentration) at the time of measurement, and a measurer. The carrier gas is a gas simultaneously injected with the smell to be measured, and for example, nitrogen or the atmosphere is used. The sample gas is a mixture of the carrier gas and the smell to be measured.

Furthermore, the above-described temperature and humidity may be acquired from set values of the object to be measured, the carrier gas, the purge gas, the sensor 10 itself, the atmosphere around the sensor 10, the sensor 10, or a device that controls the sensor 10.

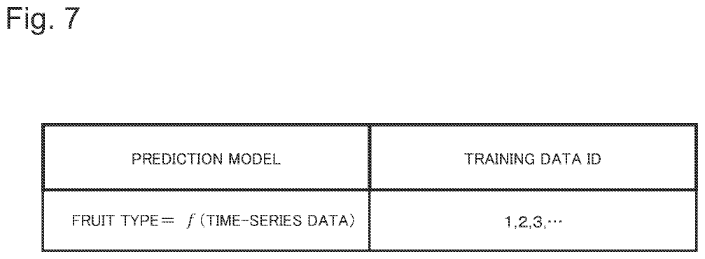

FIG. 7 is a table illustrating a correspondence relationship between the prediction model and a training data ID stored in the storage unit 2010. As illustrated in FIG. 7, the storage unit 2010 stores the prediction model and the training data ID used when learning the prediction model in association with each other. The training data ID corresponds to the ID of the smell data illustrated in FIG. 6. For example, the training data ID “1” corresponds to the ID “1” in FIG. 6. That is, the prediction model illustrated in FIG. 7 indicates that learning has been performed using the smell data of the ID “1”, ID “2”, and ID “3” in FIG. 6 as part of the training data.

Note that, in FIG. 7, a case where one prediction model is stored in the storage unit 2010 has been described as an example, but a plurality of prediction models may be stored in the storage unit 2010.

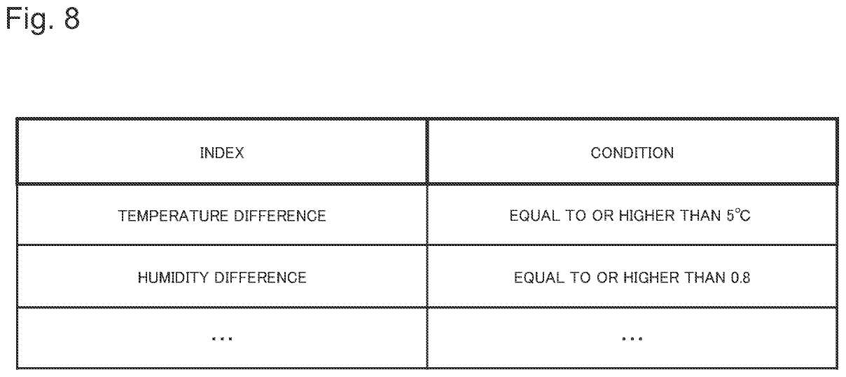

FIG. 8 is a table illustrating a condition stored in the storage unit 2010 and used for determining whether the re-learning unit 2030 performs re-learning. As illustrated in FIG. 8, an index and the condition are associated with each other. The index is an index type used to determine whether to re-learn the prediction model. The index type is a measurement environment (temperature difference, humidity difference, or the like) illustrated in FIG. 6. The condition indicates a condition for re-learning the prediction model in each index. For example, as illustrated in FIG. 8, in the case where the index is the “temperature difference”, the relevant condition is “equal to or higher than 5° C.”. That is, when the temperature difference included in the measurement environment of the smell data calculated as an index by the calculation unit 2020 is “equal to or higher than 5° C.”, the re-learning unit 2030 re-learns the prediction model. Details of index calculation processing by the calculation unit 2020 and re-learning processing by the re-learning unit 2030 will be described below.

### Processing of Calculation Unit 2020

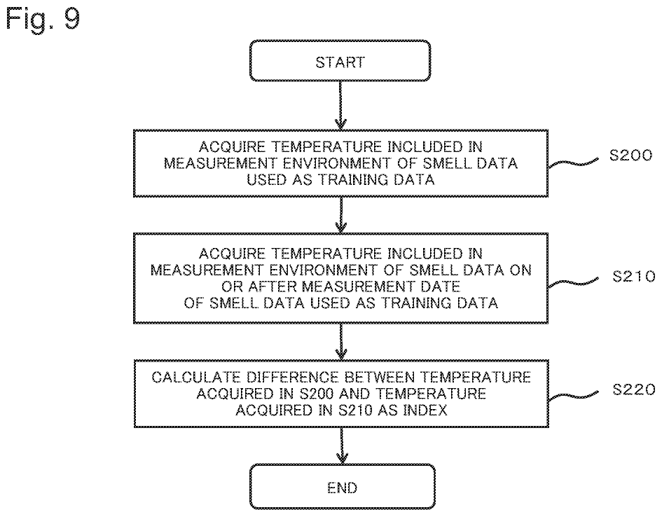

FIG. 9 is a diagram illustrating a flow of processing of the calculation unit 2020. The processing by the calculation unit 2020 will be specifically described with reference to FIG. 9. Here, a case where the calculation unit 2020 calculates the temperature difference as an index will be described as an example. Furthermore, a case where the calculation unit 2020 calculates an index for determining whether to re-learn the prediction model illustrated in FIG. 7 will be described as an example.

As illustrated in FIG. 9, first, the calculation unit 2020 acquires the temperature included in the measurement environment of the smell data used as the training data (S200). For example, the calculation unit 2020 acquires the temperature “20° C.” (FIG. 6) of the smell data with the ID “1” used as the training data.

Next, the calculation unit 2020 acquires the temperature included in the measurement environment of the smell data that is the smell data other than the smell data used as the training data and on or after the measurement date of the smell data used as the training data (S210). For example, the calculation unit 2020 acquires the temperature “10° C.” of the smell data with the ID“125” illustrated in FIG. 6.

Next, the calculation unit 2020 calculates a difference between the temperature acquired in S200 and the temperature acquired in S210 as an index (S220). For example, when the temperature acquired in S200 is “20° C.” and the temperature acquired in S210 is “10° C.”, the index is “10° C.”.

Note that, in the present example embodiment, the case where one smell data is acquired in S200 and one smell data is acquired in S210 has been described as an example. In this case, for example, the calculation unit 2020 may randomly acquire one of the smell data used for the training data or may receive designation of the smell data from the user and acquire the smell data. The same similarly applies to the smell data acquired in S210.

Furthermore, there may be a plurality of smell data acquired by the calculation unit 2020 in each of S200 and S210. In this case, the calculation unit 2020 acquires, for example, a statistical value (for example, an average value, a median value, or a mode value) of the temperatures of the plurality of smell data. The plurality of smell data may be all of the smell data used for the training data in S200 or may be the smell data designated by the user. The same similarly applies to the smell data acquired in S210.

In the present example embodiment, the case where the calculation unit 2020 acquires the temperature difference as an index has been described as an example. However, the index is not limited to the temperature difference, and may be, for example, a humidity difference or a sampling period difference.

### Processing of Re-learning Unit 2030

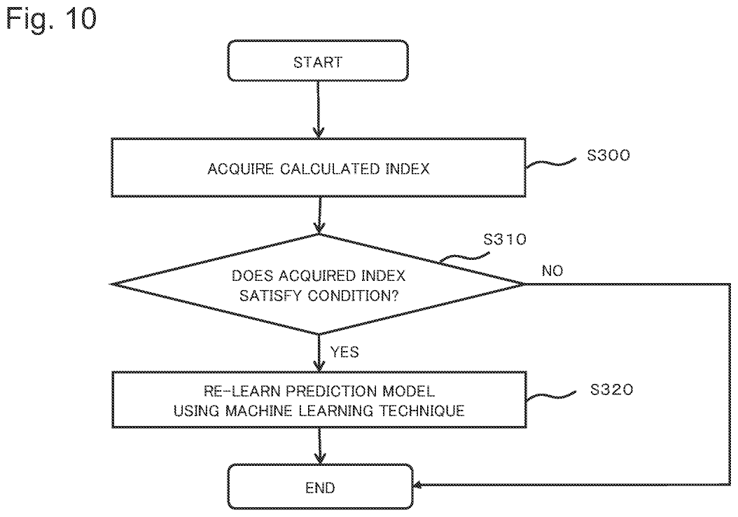

FIG. 10 is a diagram illustrating a flow of processing of the re-learning unit 2030. The processing of the re-learning unit 2030 will be specifically described with reference to FIG. 10.

As illustrated in FIG. 10, first, the re-learning unit 2030 acquires the index calculated by the calculation unit 2020 (S300). For example, the re-learning unit 2030 acquires the temperature difference “10° C.” as the index.

Next, the re-learning unit 2030 determines whether the index acquired in S300 satisfies the condition (FIG. 8) stored in the storage unit 2010 (S310). When the re-learning unit 2030 determines that the index satisfies the condition (S310; YES), the processing proceeds to S320. Otherwise, the re-learning unit 2030 terminates the processing.

When the re-learning unit 2030 determines that the index satisfies the condition (S310; YES), the re-learning unit 2030 re-learns the prediction model using a machine learning technique (for example, a technique of stochastic optimization such as a stochastic gradient descent method) (S320). For example, in the case where the index acquired by the re-learning unit 2030 is the temperature difference “10° C.”, the re-learning unit 2030 re-learns the prediction model because the condition of the temperature difference illustrated in FIG. 8 is “equal to or higher than 5° C.”.

Note that, in the present example embodiment, the case where the re-learning unit 2030 re-learns the prediction model has been described as an example, but the re-learning unit 2030 may newly generate a prediction model. In this case, the re-learning unit 2030 generates the prediction model using a new training data set. The new training data set is designated by the user, for example. As a designation method by the user, for example, a training data set may be directly input, a measurement date (or a measurement period) may be designated, a measurement environment may be designated, or a sampling method such as bagging may be designated.

### Function and Effect

As described above, the prediction model re-learning device 2000 according to the present example embodiment re-learns the prediction model in consideration of a characteristic that a behavior of the detection value of the sensor changes due to an influence of the measurement environment such as temperature and humidity. As a result, precision deterioration of the prediction model can be mitigated.

### Second Example Embodiment

Hereinafter, a second example embodiment according to the present invention will be described. The second example embodiment is different from the first example embodiment in including a feature amount acquisition unit 2040 and a calculation unit 2050 that calculates an index based on an acquired feature amount. Details will be described below.

### Example of Functional Configuration of Prediction Model Re-Learning Device 2000

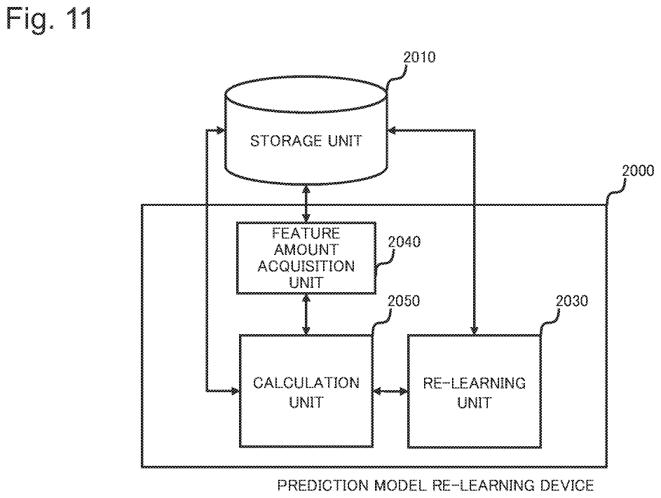

FIG. 11 is a diagram illustrating a functional configuration of a prediction model re-learning device 2000 of the second example embodiment. The prediction model re-learning device 2000 of the second example embodiment includes a feature amount acquisition unit 2040, a calculation unit 2050, and a re-learning unit 2030. The feature amount acquisition unit 2040 acquires a feature amount of time-series data included in data other than training data used for a prediction model from a storage unit 2010. The calculation unit 2050 calculates an index of the prediction model based on the acquired feature amount. An operation of the re-learning unit 2030 is similar to that of the other example embodiments, and description thereof will be omitted in the present example embodiment.

### Flow of Processing

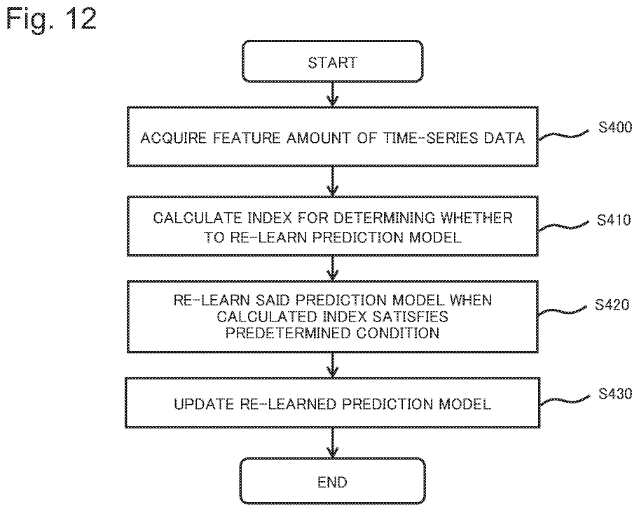

FIG. 12 is a diagram illustrating a flow of processing executed by the prediction model re-learning device 2000 of the second example embodiment. The feature amount acquisition unit 2040 acquires the feature amount of the time-series data included in data other than the training data used for the prediction model from the storage unit 2010 (S400). The calculation unit 2020 calculates an index of the prediction model based on the acquired feature amount (S410). The re-learning unit 2030 re-learns the prediction model based on the calculated index (S420). The re-learning unit 2030 stores the re-learned prediction model in the storage unit 2010 to update the prediction model (S430).

### Information Stored in Storage Unit 2010

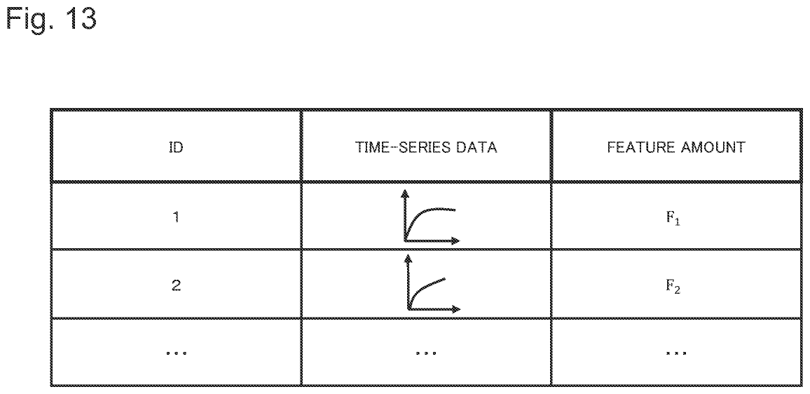

In the second example embodiment, information stored in the storage unit 2010 will be described. FIG. 13 is a table illustrating smell data stored in the storage unit 2010 in the second example embodiment.

Each record in FIG. 13 is related to smell data. Each smell data includes, for example, time-series data obtained by a sensor 10 detecting a smell and Fk that is a vector amount representing a feature amount of the time-series data. The subscript k corresponds to an ID of the smell data. Details of the feature amount will be described below.

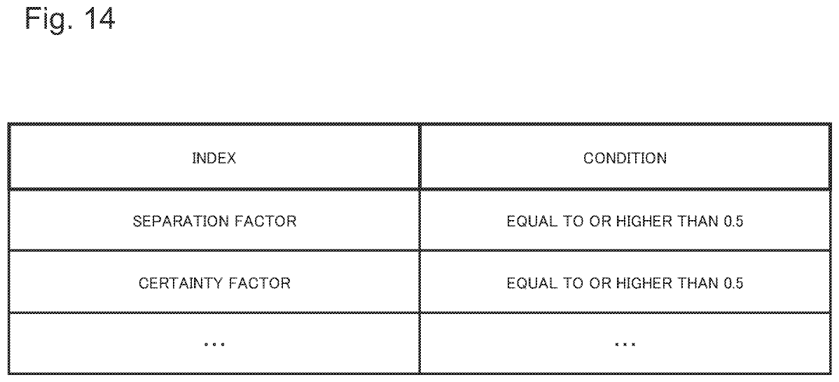

FIG. 14 is a table illustrating a condition stored in the storage unit 2010 and used for determining whether the re-learning unit 2030 performs re-learning. As illustrated in FIG. 14, an index and a condition are associated with each other. The index means an index type used to determine whether to re-learn the prediction model. The index types include a separation factor and a certainty factor. The condition indicates a condition for re-learning the prediction model for each index type. For example, as illustrated in FIG. 14, in the case where the index type is the “separation factor”, the relevant condition is “equal to or lower than 0.5”. That is, when the separation factor calculated as an index by the calculation unit 2020 becomes “equal to or lower than 0.5”, the re-learning unit 2030 re-learns the prediction model. Details of processing of calculating the separation factor and the certainty factor by the calculation unit 2020 will be described below.

### Method of Calculating Feature Amount

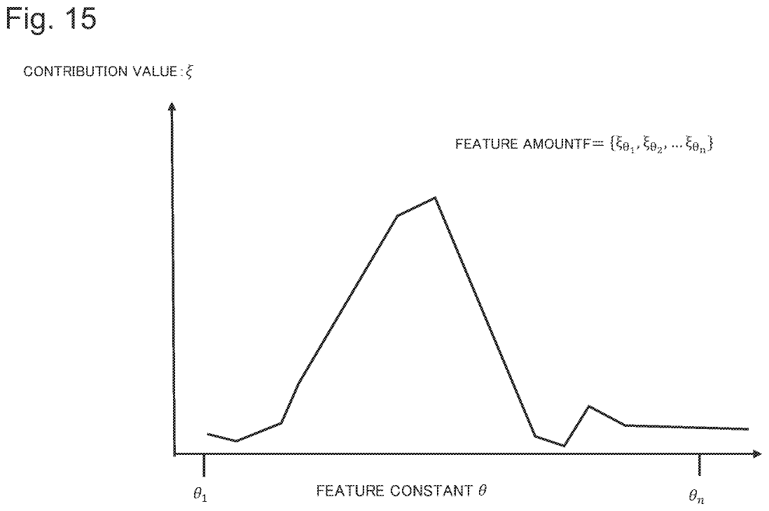

An example of a method of calculating the feature amount Fk illustrated in FIG. 13 will be described. The feature amount Fk related to each time-series data is a vector amount represented by a contribution value for each feature constant to the time-series data. Hereinafter, the feature constant and the contribution value will be described with reference to FIG. 15.

FIG. 15 is a graph illustrating the contribution value for each feature constant to the time-series data. The feature constant θ is a time constant or a velocity constant related to magnitude of a temporal change in the amount of molecules attached to the sensor 10. The feature amount Fk is a vector value represented by a contribution value ξi representing the magnitude of contribution to the time-series data y(t), for each feature constant θi (i is an integer from 1 to n; n≥1).

A method of calculating the feature constant θ and the contribution value 4 will be described. The feature amount acquisition unit 2040 decomposes the time-series data as illustrated in the following expression (1)

$$[ Math . 1 ]$$

$$y ( t ) = {{\sum }_{i = 1}}^{m} {ξ}_{i} f ( {θ}_{i} ) \qquad ( 1 )$$

In the expression (1), f is a function that differs depending on the feature constant.

When a velocity constant β is adopted as the feature constant θ, the equation (1) can be expressed as the following equation (2).

$$[ Math . 2 ]$$

$$y ( t ) = {{\sum }_{i = 1}}^{m} {ξ}_{i} {e}^{- {β}_{i} t} \qquad ( 2 )$$

When a time constant τ that is a reciprocal of the velocity constant is adopted as the feature constant θ, the equation (1) can be expressed as the following equation (3).

$$[ Math . 3 ]$$

$$y ( t ) = {{\sum }_{i = 1}}^{m} {ξ}_{i} {e}^{- t / {τ}_{i}} \qquad ( 3 )$$

### Method of Calculating Set Θ of Feature Constants θ

A method of calculating the feature constants θ₁, θ₂, . . . , θₙ (hereinafter referred to as a set Θ) will be described. The set Θ can be determined by, for example, three parameters of (1) a minimum value θ min (that is, θ₁) of the feature constant θ, (2) a maximum value θ max (that is, θₙ) of the feature constant θ, and (3) an interval ds between adjacent feature constants. In this case, the set Θ becomes Θ={θ min, θ min+ds, θ min+2ds, . . . , θ max}. Hereinafter, an example of a method for determining the above-described three parameters will be described.

(1) θ min

θ min is a constant multiple of a sampling interval Δt of the sensor 10. That is, θ min=Δt*C1 is satisfied where a predetermined constant is C1.

(2) θ max

θ max is a constant multiple of a length (the number of detection values) T of the time-series data y(t) acquired by the sensor 10. That is, θ max=T*C2 is satisfied where a value of 1 or larger is C2 in advance.

(3) ds

ds satisfies ds=(θ max−θ min)/(ns−1) where, for example, the number of the feature constants θ is ns.

In the case of using the velocity constant β as the feature constant, the minimum value θ min of the feature constant, the maximum value θ max of the feature constant, and the interval ds of the adjacent feature constants become the minimum value β min of the velocity constant, the maximum value β max of the velocity constant, and the interval Δβ of the adjacent velocity constant, respectively. Similarly, in the case of using the time constant τ as the feature constant, the minimum value θ min of the feature constant, the maximum value θ max of the feature constant, and the interval ds of the adjacent feature constants become the minimum value min of the time constant, the maximum value max of the time constant, and the interval Δτ of the adjacent time constant, respectively.

### Calculation of Contribution Vector

The feature amount acquisition unit 2040 calculates a contribution vector Ξ, which is the contribution value ξi of each feature constant θi included in the set Θ of the feature constants θ specified as described above, as the feature amount Fk. Specifically, the feature amount acquisition unit 2040 generates a detection value prediction model for predicting the detection value of the sensor 10 using the equation (1) using all the contribution values ξi (that is, the feature amount Fk, hereinafter described as “contribution vector Ξ” for the sake of description) as parameters. When generating the detection value prediction model, the contribution vector Ξ can be calculated by estimating the parameters for the contribution vector Ξ using the time-series data.

Various methods can be used for parameter estimation of the detection value prediction model. Hereinafter, an example of the method will be described. In the following description, a case where the velocity constant β is used as the feature constant is described. The parameter estimation method in the case where the time constant τ is the feature constant can be implemented by replacing the velocity constant β in the following description with 1/τ. For example, the feature amount acquisition unit 2040 estimates the parameter Ξ by maximum likelihood estimation or maximum posterior probability estimation using a prediction value obtained from the detection value prediction model and the time-series data of the detection value output from the sensor 10. Hereinafter, the case of maximum likelihood estimation will be described. For the maximum likelihood estimation, for example, a least squares method can be used. In this case, specifically, the parameter Ξ is determined according to the following objective function.

$$[ Math . 4 ]$$

$${argmin}_{Ξ ∈ {ℝ}^{m}} {\sum }_{i = O}^{T - 1} {\vert y ( {t}_{i} ) - \hat{y} ( {t}_{i} ) \vert}^{2} \qquad ( 4 )$$

In the equation (4), y\hat{ }(ti) represents a prediction value at time ti and is determined by the detection value prediction model.

The vector Ξ that minimizes the above-described objective function can be calculated using the following equation (5).

$$[ Math . 5 ]$$

$$Ξ = {( {ΦΦ}^{T} )}^{- 1} Φ Y \qquad ( 5 )$$

$${Φ}_{k , i} = \{ \begin{matrix}1 - {e}^{- {β}_{k} {t}_{i}} ( When rising ) \\ {e}^{- {β}_{k} {t}_{i}} ( When falling )\end{matrix}$$

In the equation (5), Y is a column vector obtained by transposing (y(t0), y(t1), . . . ).

Therefore, the feature amount acquisition unit 2040 calculates the parameter Ξ by applying the time-series data Y and the set Θ of feature constants={β1, β2, . . . } to the above equation (5).

Here, the meanings of “rising” and “falling” in the above equation (5) will be described. “Rising” indicates a state in which the detection value indicated by the time-series data is increasing by injecting the sample gas described in the description of the sampling period to the sensor 10. “Falling” indicates a state in which the target gas is removed from the sensor 10 by injecting the purge gas described in the description of the sampling period to the sensor 10, and the measurement value indicated by the time-series data is decreasing.

In the present example embodiment, the feature amount Fk is acquired from the “rising” time-series data and the “falling” time-series data. However, the present example embodiment is not limited thereto, and the feature amount acquisition unit 2040 may acquire the feature amount only from either the “rising” time-series data or the “falling” time-series data.

In addition, the method of acquiring the feature amount of the time-series data is not limited to the above-described method. For example, the feature amount acquisition unit 2040 may calculate the feature amount using not only the time-series data but also using the time-series data and the measurement environment. Specifically, the feature amount acquisition unit 2040 may acquire the feature amount from the time-series data and the measurement environment using a machine learning method such as a neural network.

### Index Calculation Method of Calculation Unit 2050

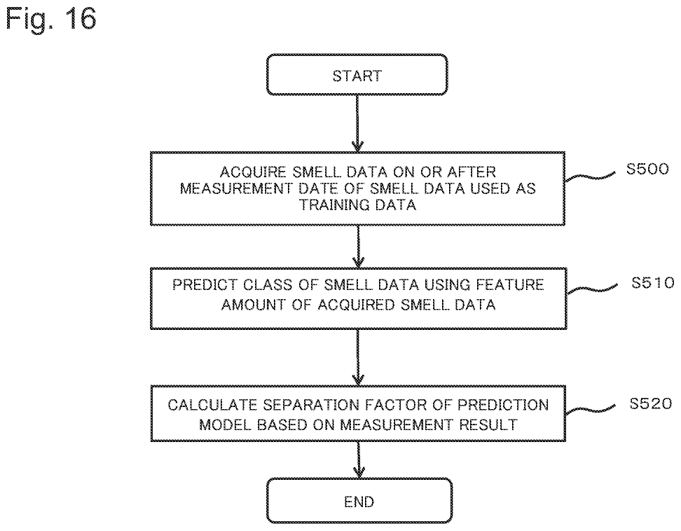

FIG. 16 is a diagram illustrating a flow of processing of a calculation unit 2050. The processing by the calculation unit 2050 will be specifically described with reference to FIG. 16. Here, a case where the calculation unit 2050 calculates the separation factor as an index will be described as an example. Details of the separation factor will be described below. Furthermore, a case where the calculation unit 2050 calculates an index for determining whether to re-learn the prediction model illustrated in FIG. 7 will be described as an example.

As illustrated in FIG. 16, first, the calculation unit 2050 acquires the smell data other than the smell data used as the training data for the prediction model and on or after measurement date of the smell data used as the training data (S500). For example, the calculation unit 2050 acquires the smell data with the ID “1” and the ID “2” illustrated in FIG. 13.

Next, the calculation unit 2050 predicts a class of the smell data using the feature amount of the smell data acquired in S500 (S510). For example, a positive class is assigned to the smell data predicted to correspond to a specific fruit type (for example, pear). A negative class is assigned to the smell data predicted not to correspond to a specific fruit type.

Next, the calculation unit 2050 calculates the separation factor of the prediction model as an index from the prediction result of each smell data (S520).

The separation factor will be described. The separation factor is expressed as, for example, a ratio of intra-class variance and inter-class variance. The intra-class variance indicates dispersion of data within a class and is represented by a sum of positive class variance and negative class variance. The inter-class variance indicates dispersion of classes in the entire data, and is calculated as a sum of the positive class variance and the negative class variance with respect to the entire data respectively multiplied by the numbers of samples of the classes. The separation factor may be directly calculated from the feature amount of the data or may be calculated from a dimensionally reduced feature amount (for example, a feature amount dimensionally reduced to a one-dimensional space).

The index calculated by the calculation unit 2050 is not limited to the separation factor that is the ratio of the intra-class variance and the inter-class variance. The calculation unit 2050 may use either the intra-class variance or the inter-class variance as an index.

In addition, the calculation unit 2050 may use the certainty factor as an index instead of the separation factor in S520.

The certainty factor will be described. For simplicity, a case where the prediction model performs binary classification will be described. The certainty factor is an index representing a degree of certainty of classification by the prediction model, and is a value obtained by a determination function as a value from 0 to 1 by, for example, a sigmoid function. At the time of learning, the prediction model is learned such that a positive class sample approaches 1 as much as possible and a negative class sample approaches 0 as much as possible. At the time of prediction, when a certainty factor larger than a threshold value (which is generally set to 0.5 in many cases) is obtained using the learned prediction model, the prediction result is output as the positive class. At this time, it can be estimated that the prediction sometimes becomes unstable as the number of data having the certainty factor near the threshold value increases, and thus, the certainty factor can be utilized as an index for re-learning.

### Function and Effect

As described above, the prediction model re-learning device 2000 according to the present example embodiment re-learns the prediction model in consideration of the feature amount of the detection value of the sensor. As a result, precision deterioration of the prediction model can be mitigated.

### Modification

A modification of the second example embodiment will be described. In the modification, the feature amount acquisition unit 2040 can acquire the feature amount after correcting the influence of the measurement environment for the time-series data.

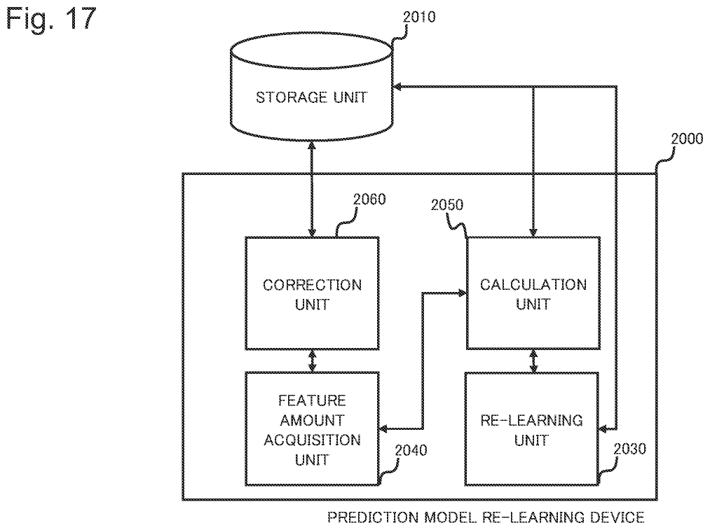

FIG. 17 is a diagram illustrating a functional configuration in the modification of the second example embodiment. The prediction model re-learning device 2000 is characterized in including a correction unit 2060 as compared with the other example embodiments. The correction unit 2060 corrects the time-series data included in data other than the training data used for the prediction model, using a correction coefficient. The feature amount acquisition unit 2040 acquires the feature amount from the corrected time-series data. Other functional configurations are similar to the operations described in another example embodiment and the second example embodiment.

An example in which the correction unit 2060 corrects the time-series data using the correction coefficient will be described. The correction unit 2060 performs correction by multiplying the time-series data y(t) by the correction coefficient. The correction coefficient relates to, for example, an individual difference of a sensory membrane of the sensor 10. The correction coefficient is calculated in advance at the time of shipment of the sensor 10, for example, and is stored in a housing provided with the sensor 10. The correction unit 2060 corrects the time-series data y(t) by acquiring the correction coefficient from the housing provided with the sensor 10.

### Third Example Embodiment

Hereinafter, a third example embodiment according to the present invention will be described. In the first and second example embodiments, the re-learned prediction model is stored and updated as it is in the storage unit 2010. However, for example, when a temporary error occurs in the measurement environment (for example, the humidity temporarily increases due to sudden heavy rain), there are some cases where update of the prediction model based on the index calculated using the measurement environment is not necessary.

Therefore, in the present third example embodiment, whether to update a prediction model is determined using a re-learned prediction model before updating the prediction model.

### Example of Functional Configuration of Prediction Model Re-Learning Device 2000

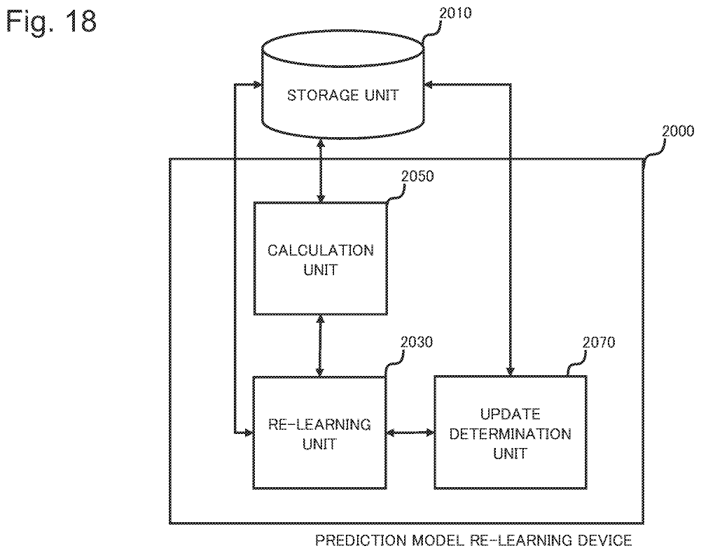

FIG. 18 is a diagram illustrating a functional configuration of a prediction model re-learning device 2000 of the third example embodiment. The prediction model re-learning device 2000 includes a calculation unit 2050, a re-learning unit 2030, and an update determination unit 2070. Since the calculation unit 2050 and the re-learning unit 2030 perform similar operations to those of the other example embodiments, description thereof is omitted here. The update determination unit 2070 performs update determination for a prediction model re-learned from the re-learned prediction model and smell data for update determination.

### Flow of Processing

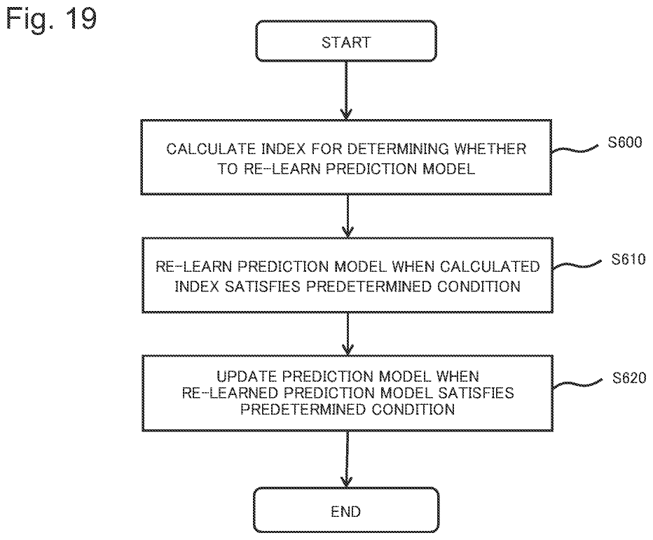

FIG. 19 is a diagram illustrating a flow of processing executed by the prediction model re-learning device 2000 of the third example embodiment. The calculation unit 2050 calculates an index for determining whether to re-learn the prediction model (S600). The re-learning unit 2030 re-learns the prediction model when the calculated index satisfies a predetermined condition (S610). The update determination unit 2070 updates the prediction model when the re-learned prediction model satisfies a predetermined condition (S620).

### Update Determination Processing of Update Determination Unit 2070

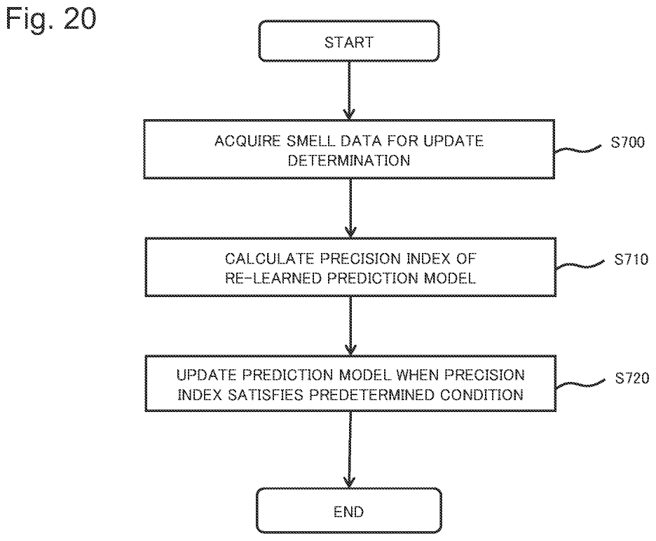

Update determination processing of the update determination unit 2070 will be described. FIG. 20 is a diagram illustrating a flow of processing of the update determination unit 2070. The processing by the update determination unit 2070 will be specifically described with reference to FIG. 20. Here, a case where the update determination unit 2070 calculates an index for determining whether to re-learn the prediction model illustrated in FIG. 7 will be described as an example.

First, the update determination unit 2070 acquires the smell data for update determination (S700). The smell data for update determination is smell data different from the smell data used for calculating the index for determining whether to re-learn the prediction model. A specific example of the smell data for update determination will be described with reference to FIG. 6. When the measurement date of the training data of the prediction model is “2016/10/15” and the smell data with the ID “125” is used for index calculation, the update determination unit 2070 acquires (1) the smell data with an ID different from “125” as the smell data for update determination.

The update determination unit 2070 may receive designation of a condition for one or more of the sensor ID, the measurement date and time, the measurement environment, and the object to be measured illustrated in FIG. 6 in addition to the above-described condition (1) and acquire the smell data including the designated condition as the smell data for update determination.

The description returns to the description using FIG. 20. The update determination unit 2070 calculates a precision index of the re-learned prediction model using the acquired data for update determination (S710). The precision index is, for example, Precision, Recall, Specificity, F-number, Accuracy, and AUC.

Note that, here, an example of the precision index in the case where the prediction model is a determination model has been described. In the case where the prediction model is a regression model, the precision index is, for example, a determination coefficient, a mean square error, and a mean absolute error.

Next, when the precision index calculated in S710 satisfies a predetermined condition, the update determination unit 2070 stores the re-learned prediction model in the storage unit 2010 to update the prediction model (S720). The predetermined condition is, for example, whether the precision index calculated in S710 is equal to or larger than the threshold value. The threshold value of the precision index may be stored in advance in the storage unit 2010 or may be input from the user.

### Function and Effect

As described above, since the prediction model re-learning device 2000 according to the present example embodiment determines whether to update the prediction model according to the precision of the re-learned prediction model, the prediction model re-learning device 2000 can avoid unnecessary update of the prediction model.

Note that the invention of the present application is not limited to the above-described example embodiments as they are, and can be embodied by modifying the constituent elements without departing from the gist thereof at an implementation stage. Furthermore, various inventions can be formed by appropriately combining a plurality of constituent elements disclosed in the above example embodiments. For example, some constituent elements may be deleted from all the constituent elements shown in the example embodiments. Moreover, the constituent elements of different example embodiments may be appropriately combined.

### REFERENCE SIGNS LIST

10 Sensor
20 Time-series data
1000 Computer
1020 Bus
1040 Processor
1060 Memory
1080 Storage device
1100 Input/output interface
1120 Network interface
2000 Prediction model re-learning device
2010 Storage unit
2020 Calculation unit
2030 Re-learning unit
2040 Feature amount acquisition unit
2050 Calculation unit
2060 Correction unit
2070 Update determination unit

What is claimed is:

1. A prediction model re-learning device comprising:
at least one memory storing instructions; and
at least one processor configured to access the at least one memory and execute the instructions to:
calculate an index for determining whether to re-learn a prediction model for a smell based on data related to smell detection by a sensor; and
re-learn the prediction model in a case where the calculated index satisfies a predetermined condition.

2. The prediction model re-learning device according to claim 1, wherein
the data related to smell detection by a sensor indicates a measurement environment of the smell by the sensor, and
the at least one processor is further configured to execute the instructions to:
calculate a difference between a measurement environment of training data of the prediction model and a measurement environment of data other than the training data as the index.

3. The prediction model re-learning device according to claim 2, wherein
the measurement environment of the smell includes at least either temperature or humidity.

4. The prediction model re-learning device according to claim 1, wherein
the data related to smell detection by a sensor indicates a feature amount of data other than training data of the prediction model, and
the at least one processor is further configured to execute the instructions to:
calculate the index based on the feature amount and the prediction model.

5. The prediction model re-learning device according to claim 1, wherein
the data related to smell detection by a sensor indicates a feature amount of training data of the prediction model and a feature amount of data other than the training data, and
the at least one processor is further configured to execute the instructions to:
calculate the index based on the feature amount of training data of the prediction model and the feature amount of data other than the training data.

6. The prediction model re-learning device according to claim 4, wherein
the at least one processor is further configured to execute the instructions to:
correct a detection value of a smell by the sensor based on a correction coefficient calculated from an individual difference of the sensor, and
the feature amount is a feature amount of the corrected detection value.

7. The prediction model re-learning device according to claim 1, wherein
the at least one processor is further configured to execute the instructions to:
perform update determination for a prediction model re-learned from the re-learned prediction model and the data related to smell detection for update determination.

8. The prediction model re-learning device according to claim 2, wherein
the data other than training data is data on or after a measurement date of data used as the training data.

9. A prediction model re-learning method comprising:
by a computer,
calculating an index for determining whether to re-learn a prediction model for a smell based on data related to smell detection by a sensor; and
re-learning the prediction model in a case where the calculated index satisfies a predetermined condition.

10. A non-transitory computer-readable program recording medium recording a program for causing a computer to execute:
processing of calculating an index for determining whether to re-learn a prediction model for a smell based on data related to smell detection by a sensor; and
processing of re-learning the prediction model in a case where the calculated index satisfies a predetermined condition.
